(19) United States
(12) Patent Application Publication
(10) Pub. No.: US 2009/0182041 A1
(43) Pub. Date: Jul. 16, 2009

(54) CONTROL OF CANCER WITH ANNONACEOUS EXTRACTS

(75) Inventors: Jerry Loren McLaughlin, West Lafayette, IN (US); Gina Bellessa Benson, Provo, UT (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: NATURE'S SUNSHINE PRODUCTS, INC., Provo, UT (US)

(21) Appl. No.: 12/353,853

(22) Filed: Jan. 14, 2009

Related U.S. Application Data
(62) Division of application No. 10/717,746, filed on Nov. 20, 2003, now abandoned.
(60) Provisional application No. 60/428,602, filed on Nov. 22, 2002.

(51) Int. Cl. A61K 31/341

(52) U.S. Cl. 514/473

(57) ABSTRACT

A crude extract composition derived from the plant family Annonaceae and the method for producing the crude extract composition. Medicinal benefits of the extract include tumor regression and the reduction of tumor antigen levels.

### CROSS-REFERENCE TO RELATED APPLICATIONS

This patent application is a divisional of now pending U.S. patent application Ser. No. 10/717,746, filed Nov. 20, 2003 and entitled CONTROL OF CANCER WITH ANNONACEOUS EXTRACTS, which claims benefit to U.S. Provisional Patent Application Ser. No. 60/428,602, filed on Nov. 22, 2002, all of which are incorporated herein by reference.

### BACKGROUND

To be effective, chemotherapeutic agents must eradicate enough tumor cells for the body's immune defenses to eliminate any remaining tumor cells. Difficulties with most of the chemotherapeutic drugs emanate from their concurrent eradication of normal healthy cells, including those responsible for immunity. Additionally, the eventual development of drug resistance by the tumor cells often renders chemotherapy useless and futile after a period of remission.

While adenosine triphosphate (ATP) is a precursor to the nucleotides needed to produce deoxyribonucleic acid (DNA) and ribonucleic acid (RNA), and is also the major source of intracellular biochemical energy, the inhibition of ATP production has been deemed as too general a mechanism for systemic cancer chemotherapy. It has been regarded that all cells require ATP, and thus, ATP inhibitors would be simultaneously cytotoxic to essential tissues as well as cancer cells.

The endogenous molecular biology of cancer cells, however, is now understood to involve autocrine and paracrine secretion of insulin and insulin-like growth factors (IGF-I & II) that subserve enhanced energy production and growth stimulation, respectively, in these cells (Ayre et al., 2000). Breast cancer cells have an average of seven times more insulin receptors (Papa et al., 1990) and ten times more IGF receptors (Cullen et al., 1990) than normal breast and other tissue cells within the host. Thus, these cancer cells can take up glucose seventeen times faster than normal cells, and, it must be presumed that, they can also utilize glucose seventeen times faster than normal cells, therefore producing ATP at a faster rate.

The depletion of ATP and related nucleotides (all of which are precursors of DNA and RNA) has been demonstrated in vitro in human leukemic cells (Fotopoulos) and the result is an upset of cell timing with subsequent apoptosis (programmed cell death) as demonstrated in malignant B cells (Geahlen). The increase in metabolic activity and cell membrane permeability to glucose makes tumor cells more susceptible than normal cells to the effects of ATP depletion.

The paw paw tree, Asimina triloba (L.) Dunal (Annonaceae), is native to the eastern United States. The major active compounds in the Annonaceae family are called annonaceous acetogenins (or acetogenins). These are long chain fatty acid derivatives (C-32 and C-34) that terminate in an α, β-unsaturated γ-lactone ring, and they typically contain from zero to three tetrahydrofuran (or tetrahydropyran) rings in the chain. The paw paw tree contains over 50 active acetogenins. Several related tropical and subtropical species in the Annonaceae family (e.g., species in the annonaceous genera Annona, Asimina, Goniothalamus, Rollinia, Uvaria, and Xylopia) have yielded an additional 350 compounds in this class.

Biologically annonaceous acetogenins are powerful inhibitors of mitochondrial and cytoplasmic production of ATP. These compounds selectively inhibit cancer cells (vs. normal cells) and in vivo tumors, and also thwart multiple drug resistant (MDR) tumor cells that are dependent on ATP-driven efflux pumps. The annonaceous acetogenins slow or stop ATP production at mitochondrial complex I (NADH: ubiquinone oxidoreductase) and at the NADH oxidase of tumor cell membranes. Tumor cells are typically metabolically more active and have increased membrane permeability to glucose, making them more susceptible than normal cells to the effects of the acetogenins.

Vascular endothelial growth factor, which induces angiogenesis, requires ATP (Satake et al., 1998), and angiostatin inhibits ATP synthase (Moser et al., 1999) as it blocks angiogenesis. Thus, ATP depletion helps to block the growth of new vessels to nourish tumors. In addition, drug resistance in tumors is often due to the development of an ATP-dependent efflux pump, which extrudes the chemotherapeutic agent back into the extracellular matrix/bloodstream, allowing it to harm healthy cells as well as non-drug resistant tumor cells. This ATP-dependent efflux pump is thwarted by the acetogenins as it is driven by ATP, and ATP production is slowed by the acetogenins.

Bullatacin, asimicin and trilobacin (annonaceous acetogenins), in substantially purified form, are the most powerful inhibitors known of complex I in the electron transport system in mitochondria (Lewis et al., 1993; Hollingworth et al., 1994; Ahammadsahib et al., 1993; Landolt et al., 1995; Alfonso et al., 1996; He et al., 1997), and they also inhibit the NADH oxidase found in the plasma membranes of tumor cells (Morre et al., 1995). Their net effect is depletion of ATP levels. In vivo studies, against murine leukemia, myeloma, and human ovarian carcinoma in athymic mice, attest to the biological effectiveness of several of the acetogenins in pure form (Ahammadsahib et al., 1993; Gu et al., 1995).

### SUMMARY OF THE INVENTIONS

The present inventions demonstrate, unexpectedly, that complex mixtures of annonaceous acetogenins, as crude extracts (as opposed to conventional substantially purified forms), are biologically active against a wide range of tumor types in cancer patients. The crude extracts also thwart development of resistance to chemotherapeutic agents. As such, an improved and simplified method has been developed for extracting crude extracts of annonaceous acetogenins. The crude extracts of annonaceous acetogenins provide medicinal uses, such as improved and inexpensive treatments for cancer.

### BRIEF DESCRIPTION

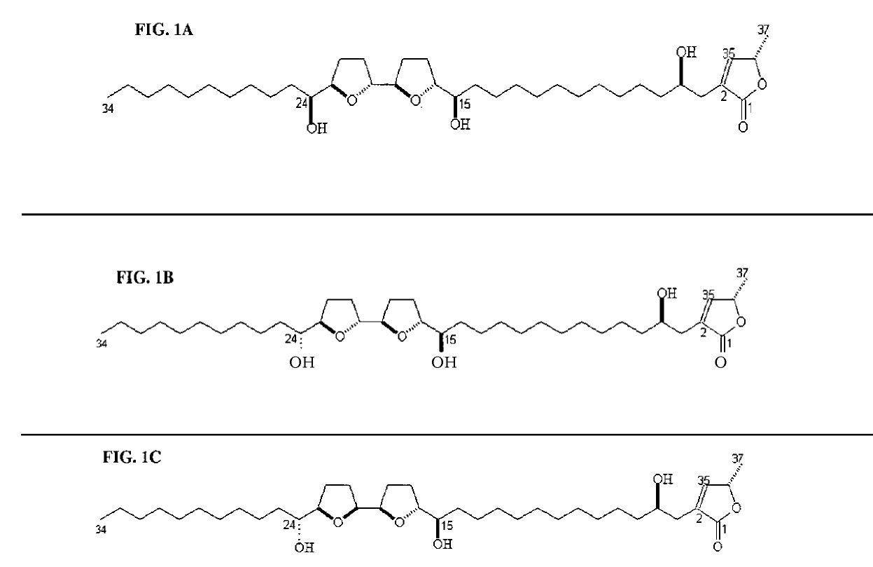

FIG. 1 illustrates the complete chemical structures with their absolute stereochemistries defined for the annonaceous acetogenins: FIG. 1A-bullatacin, FIG. 1B-asimicin, and FIG. 1C-trilobacin.

### DETAILED DESCRIPTION

Substantially purified forms of annonaceous acetogenins have been used to inhibit specific cancer cells and thwart multiple drug resistant tumor cells. These purified forms, however, are difficult and costly to manufacture. In addition, the purified forms may be limited to include one or a very few acetogenins, and therefore provide specificity towards a limited number of cancer cells. It has been discovered that crude extracts provide a more cost effective way of obtaining a large number of annonaceous acetogenins with broad application across a variety of cancers.

One embodiment includes crude extracts of the twigs of the paw paw tree, Asimina triloba. Such crude extracts are an effective supplement to chemotherapy and, even alone, exert useful anti-tumor effects on a variety of cancers. In an alternative embodiment, a crude extract is derived from the unripe fruit, seed, bark and/or other bioactive part of the paw paw tree. In other alternative embodiments, one or more twig, unripe fruit, seed, bark and/or other bioactive part, or any combination thereof, of annonaceous species in the genera Asimina, Annona, Goniothalamus, Uvaria, Disepalum, Xylopia, and Rollinia may be used to prepare a crude extract in accordance with the present inventions.

Preparation of the Crude Extract

The bioactive components of the paw paw have been isolated and identified individually by bioassay-directed fractionation guided by the brine shrimp lethality test (BST). Using this bioassay to guide fractionation, a complex mixture of over 50 annonaceous acetogenins has been found in paw paw tree. The BST, followed by high performance liquid chromatography/tandem mass spectrometry (LC/MS/MS), demonstrates that concentrations of the annonaceous acetogenins are maximal in the months of May to June. The bioactive components represented in the crude extract are particularly concentrated in the twigs of the paw paw tree. In alternative embodiments, other species of the Annonaceae family may be used to produce the crude extract.

In one embodiment, about 3000 pounds of dried twigs of Asimina triloba are obtained in the month of May. Preferably, only those twigs that are ½ inch or less in diameter are collected. The twigs are dried in a forced air drier at about 50° C. (+/−0-200° C.) and pulverized in a chipper/shredder through a ¼ inch sieve before being introduced into a percolator. Extraction using the percolator is initiated with hot (city) water at one gallon per pound of twigs. After the twigs have soaked for eight hours, the water is drained and discarded to remove the benzyltetrahydroisoquinoline alkaloids. This water extraction is repeated three additional times. The damp mass is then extracted four more times with 95% ethanol, in a similar manner. The ethanolic extract is concentrated, in vacuo, at about 50° C. (+/−0-200° C.), to form a syrup. Upon sitting, a water layer develops and is removed and discarded, leaving the crude extract. The crude extract is standardized for 0% moisture and an LC₅₀ value of 0.5 ppm in the BST. Preferably, the extract will contain from ca 10-40% moisture, and the LC₅₀ value will range from 0.2-0.8 ppm.

One of skill in the art will appreciate that adjustments are made in the weight to accommodate the standard values. The mixture of acetogenins is monitored chemically using LC/MS/MS (Gu et al., 1999) to be assured of the presence of certain major acetogenins (e.g., FIG. 1 A-C) as marker compounds.

In one embodiment, the extract is formulated into servings for oral administration in a capsule or tablet, but one of skill in the art will recognize that alternative forms of administration, including tinctures, may also be utilized. Capsules or tablets preferably contain an excipient for the crude extract as well as conventional fillers and tableting agents.

Treatment Using the Crude Extract

In another embodiment, a method for administering the crude extract includes ingesting capsules containing 12.5 mg of the crude extract four times daily (QID) with food. This method is tolerated well in humans and induces tumor reduction. In general, however, this amount (50 mg per day) is based on a 70-kilogram person. Adjustments can be made for those weighing more or less. Children, for example, may decrease the dose by 25 to 50%. As each patient's tolerance level will be different, it is suggested to start slowly and gradually increase the dose. In addition, dosage adjustments may be required for veterinary applications.

In yet another embodiment, a method for determining a patient's tolerance includes ingesting one 12.5 mg capsule on day one, two capsules on day two, and so forth, building up to four capsules. Some patients may not tolerate 50 mg of the crude extract per day while others may take in excess of 200 mg (up to 500 mg) per day without adverse side effects. Thus, preparation and dosages may differ. It has also been noted that taking the extract with food may lessen the occurrence of nausea or stomach upset.

### TREATMENT EXAMPLES

A clinical study was performed to test the crude extract on tumor antigen levels and tumor regression. Capsules including the crude extract at 12.5 mg with excipients were administered four times daily (QID) with food for a study period of at least 180 days. Blood collections were taken over the course of the study at days zero, 60, 120 and 180 to evaluate specific blood serum antigen levels. Day zero blood collection provided a baseline count.

Volunteer participants were recruited from physicians and other healthcare providers whose patients agreed to participate. Only participants diagnosed with clinical cancer were included, and many participants had stage four cancer that was deemed terminal. Those who were concurrently undergoing chemotherapy or radiation were included, along with those who had not had long-term success with chemotherapy/radiation and those who had refused these options due to their known devastating effects on the immune system and general well-being.

Approximately 100 participants enrolled in the study. Each participant signed an informed consent and medical records release statement. Participants were monitored by their healthcare provider for any adverse effects as well as for positive effects. An in-house Institutional Review Board, comprised of outside professionals, reviewed the protocols and found no concern regarding the safety of the participants. The healthcare providers were requested to discuss any adverse events with their patients. Additionally, the providers contacted Nature's Sunshine Products, Inc. to report any adverse event within 24 hours of administration of the capsules. If the providers were unable to be contacted, the participant was able to call an after hours number printed on the informed consent form. The study coordinator compiled the signed consent forms from the participants and recorded adverse events, compliance, positive results, dates of treatment, marker determinations and other concerns the participant or healthcare provider may have had.

The capsules containing the crude extract unexpectedly exhibited significant benefit to the participants by stabilizing and reversing the progression of clinical cancer, as illustrated in the following examples.

### Example A

Individuals with bone cancer have elevated levels of alkaline phosphatase in their blood. The level of alkaline phosphatase is used to monitor progress of the disease, wherein the normal range is 0-136. A participant suffering from bone cancer had undergone treatments in 2002 including radiation in the spinal area. A blood test taken in September 2002 yielded a level of alkaline phosphatase of 327. The participant started taking four 12.5 mg crude extract capsules per day in November 2002. By December 2002 the level of alkaline phosphatase slightly decreased to 242 and in February 2003 the level decreased to 144. The level has remained stable (between 144 and 150) since February. According to the participant's physician, the cancer is contained and not doing further damage as indicated by the stable level of alkaline phosphatase. The participant reports to have more energy and stamina while taking the capsules.

### Example B

A participant suffering from a bone tumor in the neck participated in the study. On July 30, 2002, the bone tumor was measured as a 7 mm cavity with a 5 mm mass, according to x-rays. The participant started taking crude extract capsules in September 2002 without any additional treatment. An x-ray taken on March 13, 2003, showed a significant decrease in tumor size such that the cavity was measured to be 4.5 mm with a 3 mm mass.

### Example C

Individuals with breast cancer may have levels of CA2729 (tumor marker) above 15. Blood tests evaluating the level of CA2729 can indicate how a breast cancer tumor is responding to treatment. A participant suffering from breast cancer has not undergone any conventional treatments since being diagnosed. She has taken crude extract capsules since November 2002. Blood tests taken on September 12, 2002, and December 3, 2002, yielded a level of 24.6. By March 2003 the level of CA2729 was within normal range. The tumor size has also reduced.

### Example D

A participant suffering from breast cancer has not undergone any conventional treatments since being diagnosed. She has taken crude extract capsules since October 2002. She reports that pain in the affected breast has decreased and the non-cancerous fibrocystic lumps have reduced in size. Her doctor reports she has been doing “remarkably well” considering that she has not had surgery, chemotherapy or radiation. She says that she feels good and has gained some weight following a significant loss.

### Example E

A participant suffering from breast cancer underwent chemotherapy treatments while taking crude extract capsules. The chemotherapy treatments continued for seven months. The tumor almost completely disappeared. The participant had surgery to remove any traces of the cancer, resulting in the removal of 14 axillary lymph nodes that showed no metastatic cancer. The surgery was followed by radiation. The participant continues to be cancer free.

### Example F

A participant suffering from stage four breast cancer started taking crude extract capsules, without changing any other treatment protocol. After just six weeks of taking the capsules, a 50% percent reduction in the level of CA2729 resulted, dropping from 160 to 80. The size of the tumor also reduced significantly.

### Example G

The carcinoembryonic antigen or CEA (tumor marker) is not normally found in the blood of adults. Those with lung cancer have elevated levels. The presence and level of CEA is used to determine how widespread a cancer has grown and also to determine the success of a treatment. A participant suffering from stage four lung cancer had undergone two years of chemotherapy without success. During this time, the participant was limited to a wheelchair or bedridden. Within two months of taking crude extract capsules his tumor markers improved, showing a decrease from 275 to 222. The participant had a weight gain of five pounds and did not suffer from side effects of the crude extract capsules. The participant is now able to walk on his own.

### Example H

A participant suffering from stage four melanoma started taking crude extract capsules in November 2002. The melanoma had previously metastasized to the lungs causing great difficulty while breathing. The participant experienced easier breathing within days of taking crude extract capsules. The participant has since been able to get out of bed and even progressed to riding a bike, walking uphill and working on a farm. In addition, two fatty tumors on the participant's arm have also decreased considerably in size.

### Example I

The prostate specific antigen or PSA is an indicator of the growth of prostate cancer and is also used to determine the success of a treatment, measured through blood tests. A normal PSA level for a 51-60 year old individual is 0-3.5. A 56 year old participant suffering from prostate cancer, that was confirmed by biopsy, started taking four 12.5 mg crude extract capsules per day in October 2002. His PSA levels dropped from 3.85 on October 2002 to 2.08 on December 2002. This participant continued to take crude extract capsules until April 2003.

### Example J

A participant suffering from stage four metastasizing prostate cancer started taking crude extract capsules. There was a distinct reduction in the tumor masses within six weeks of taking the capsules, although he was taking only two (instead of four) capsules or 25 mg (instead of 50 mg) per day. A subsequent CT scan showed a 25% reduction in the tumors. The participant's PSA level is remaining constant.

The examples listed above particularly show the efficacy of the crude extract. Table 1 is a complete list of the experiment results.

TABLE 1
Progress of patients with clinical cancer taking capsules containing crude extract.

| Number | Cancer Type | Comments |
|---|---|---|
| 1 | Bone cancer | Started at the end of January. December 2002 |
| | | Alk-Phos test was 242. Feb. 22, 2003 test was |
| | | 144. (Normal range is 0-136, alkaline |
| | | phosphatase is elevated in bone cancer.) Alk- |
| | | Phos results continue to stay constant and within |
| | | normal range. |
| 2 | Bone tumor in the | Started paw paw in September 2002. On Jul. 30, 2002 |
| | neck | the bone scan showed a 7 mm cavity and a 5 mm |
| | | mass on the neck. By Mar. 13, 2003 the scan |
| | | showed a decrease in tumor size to a 4.5 mm |
| | | cavity with a 3 mm mass. |
| 3 | Brain cancer | Started the paw paw in January 2003. He has |
| | | continued the product but the tumor has shown a |
| | | small amount of growth. Now doing low dose |
| | | chemo along with the paw paw. |
| 4 | Brain cancer | Took the product for only 3 weeks but tolerated it |
| | | well. |
| 5 | Brain cancer | Started the paw paw in January 2003. Has continued |
| | | to use the product. MRI has been scheduled but |
| | | the results have not been obtained. |
| 6 | Brain cancer | Started the paw paw in February 2003. An MRI on |
| | | Apr. 16, 2003 showed change in tumor, center |
| | | appeared liquefied. The tumor was removed |
| | | surgically Apr. 17, 2003 and the removed mass |
| | | is being tested. |
| 7 | Brain cancer- | Was unable to tolerate product. |
| | anoplastic | |
| | astocytoma | |
| 8 | Brain cancer- | Started in March 2003; tolerating it well and has |
| | atypical arabdoid | been feeling well. |
| 9 | Breast cancer | Started the paw paw in January 2003 but |
| | | discontinued in March due to development of |
| | | myengioma cancer. |
| 10 | Breast cancer | Started the paw paw in July 2002. Has been |
| | | taking up to 16 capsules per day. |
| 11 | Breast cancer | Started the paw paw in October 2002. She has been |
| | | doing well since then. |
| 12 | Breast cancer | Started taking product in January but was in and |
| | | out of the hospital. Just recently started taking |
| | | product again in April. |
| 13 | Breast cancer | Pain in breast has decreased, fibrocystic lumps |
| | | have reduced in size. MD reports she has been |
| | | doing “remarkably well” as she has not done |
| | | conventional treatments. |
| 14 | Breast cancer | CA 27.29 (breast cancer antigen) has been |
| | | consistent since starting the paw paw. It was at |
| | | 24.6 on Sep. 12, 2002 and 24.6 on |
| | | Dec. 3, 2002. In March all the blood tests |
| | | were within normal range. The tumor size has |
| | | been reduced to the size of a pea. |
| 15 | Breast cancer | With one round of chemo taken conjunctively with |
| | | the paw paw her tumors disappeared almost |
| | | completely. She followed up with several more |
| | | rounds of chemo. The remaining 3 fragments |
| | | were removed surgically. In January 2003 the |
| | | checkup was clear. 14 lymph nodes were |
| | | scanned and all are clear. |
| 16 | Breast cancer | Started the paw paw in January 2003. Waiting for |
| | | results on CAT scan, bone scan, and blood work. |
| 17 | Breast cancer | October 2002 start. CA 27.29 on Dec. 3, 2002 |
| | | was 32. Stopped product in February and is |
| | | doing chemotherapy for bone metastases. |
| 18 | Breast cancer | Started the paw paw in October 2002. Her CA 27.29 |
| | | has stayed constant for a few months at 47 but |
| | | both wcc and rbc counts have increased. On |
| | | Mar. 19, 2002 the CA 27.29 decreased to 34 |
| | | (lowest in 3½ years) and scans showed one |
| | | tumor is gone and the other is barely visible. |
| 19 | Breast cancer- | February 1 start. Waiting on PET scan results. PET |
| | Adenocarcinoma | scan was done because Jan CAT scan showed |
| | | lesions on the liver. PET scan of the liver and |
| | | bone normal but existing breast cancer remains, |
| | | also in left axillary. |
| 20 | Breast cancer- | Within 6 weeks taking the paw paw saw a 50% |
| | stage 4 | reduction of tumor marker levels that went from |
| | | 160 to 80. Currently in stable condition. |
| 21 | Breast cancer- | Currently in stable condition. |
| | stage 4 | |
| 22 | Breast cancer- | November 2002 start. Took paw paw for a couple |
| | stage 4 | months but did not continue due to new |
| | | circumstances: has chemo induced leukemia, |
| | | will be doing stem cell transplant. |
| 23 | Breast/lymph nodes | She has taken the product steadily since |
| | | September 2002. She is doing well after finishing |
| | | chemotherapy and radiation treatments in |
| | | February. |
| 24 | Carcinoid, malignant | Passed away, small bowel obstruction. |
| 25 | Cervical cancer | End of September 2002 start date. Doing really |
| | | well and feeling very well; energy levels have |
| | | increased. |
| 26 | Cervical cancer- | Started the paw paw in January 2003. She has been |
| | Small cell | feeling well and the blood cell count has been |
| | | very good. |
| 27 | Colon cancer | According to the MD the paw paw helped her for |
| | | 1-2 months but she expired. |
| 28 | Colon cancer | Started in mid-October. Currently not taking, had |
| | | too much nausea with chemotherapy. |
| 29 | Colon cancer | Stable colon cancer; CEA (carcinoembryonic |
| | | antigen) falling. |
| 30 | Colon cancer | Started the paw paw in January 2003. She |
| | | reports that she has a lot more energy and is |
| | | feeling better. She also underwent 2 rounds of |
| | | chemotherapy. |
| 31 | Colon cancer | Has been taking the paw paw since November |
| | | 2002. The CEA levels have dropped from 29 to 3 |
| | | and are remaining stable. |
| 32 | Colon cancer | January 2003 start. Sending in blood work. |
| 33 | Colon cancer- | Started the paw paw in January 2003. He has seen a |
| | stage 4 | small increase in the tumor size but during the |
| | | past couple of months he has been sick with |
| | | other ailments and has not been taking product |
| | | every single day. |
| 34 | Esophageal | November 2002 start. Has had various surgeries and |
| | | treatments but continues taking paw paw. |
| 35 | Leukemia | Oct. 26, 2003 started the paw paw but he had |
| | | some nausea/vomiting so he may not have |
| | | continued the product. His cancer was |
| | | diagnosed terminal and he passed away in |
| | | January. |
| 36 | Leukemia | Last white cell count slightly down from 297K to |
| | | 279K. |
| 37 | Liver cancer | Started the paw paw in January 2003 but she later |
| | | had some unrelated stomach problems and |
| | | stopped taking the product. |
| 38 | Liver cancer | Has been taking paw paw since January 2003 |
| | | and doing well. Will be reevaluated by physician |
| | | soon. |
| 39 | Liver cancer | December 02 start and overall is doing well. However, |
| | | most recently she was unavailable for update |
| | | because she is out of town for extended time |
| | | period. |
| 40 | Lung cancer | November 2002 start. Has done chemo; continued with |
| | | paw paw. |
| 41 | Lung cancer | Did 6 chemotherapies. MRI looked good in lung, |
| | | brain, hip. Will start paw paw again after |
| | | treatments. |
| 42 | Lung Cancer | Started the paw paw in January 2003. Since this time |
| | | has been feeling very good and has increased |
| | | energy and lung capacity. Able to walk 1.5 |
| | | miles/day and golf. |
| 43 | Lung cancer | January 2003 start; CAT scan scheduled for May 1, |
| | | 2003. |
| 44 | Lung cancer-stage 4 | In 2 months CEA marker decreased from 275 to |
| | | 222, has refractory lung cancer (resistant to 2 |
| | | years of chemotherapy). With paw paw |
| | | supplementation has gained 5 lbs and is now |
| | | ambulatory whereas before was chair or |
| | | bedridden. |
| 45 | Lung cancer-stage 4 | Started taking the capsules in November 2002. |
| | | Condition is stable and has been gaining weight. |
| | | Patient reports that is less uncomfortable and |
| | | there has been some tumor shrinkage. |
| 46 | Lung Cancer-Non | Clinically improving: gained weight and feeling |
| | Small Cell Squamous | good, no shortness of breath. |
| 47 | Lymphoma, Non | Started the capsules in August 2002. Diagnosed |
| | Hodgkin's | with cancer for 6½ years and had 3 rounds of |
| | | chemo. Blood tests showed a stable condition: |
| | | Sep. 12, 2002 test reported 34.1 white cell count, 67.2% |
| | | lymphocytes, Oct. 17, 2002 test reported 11.2 wcc, |
| | | 30.0% lymphocytes with other white cells in more |
| | | normal ranges as well. (3.5-12 is normal range |
| | | for wcc, lymphocytes should normally be |
| | | 16-43%). Was continuing to do well in January 2003 but |
| | | unfortunately by March 2003 condition was |
| | | deteriorating with a tumor behind the eye. |
| | | Passed away on Mar. 20, 2003. |
| 48 | Lymphoma, | This participant continued to be stable in the |
| | Waldenstrom's | disease and shows subjective improvement. |
| | Macroglobulinemia | |
| 49 | Lymphoma-stage 4 | Started the paw paw in August 2002. Non- |
| | | Hodgkin's lymphoma is in stable condition. It is |
| | | low grade and has shown improved nodes and |
| | | Beta 2 microglobules. |
| 50 | Melanoma | Started the paw paw in January 2003. Has had some |
| | | lymph glands and a toe removed but continues |
| | | taking the paw paw and is feeling very well. |
| 51 | Melanoma-stage 4 | Began taking paw paw in October 2002. Within |
| | | days was feeling much better. The melanoma |
| | | has metastasized to the lungs and previously |
| | | patient had great difficulty breathing. Since |
| | | starting the paw paw feeling very good and has a |
| | | much easier time breathing. Has been able to |
| | | get out of bed and even progressed to doing |
| | | activities like riding a bike and walking uphill. |
| | | Interestingly, two fatty tumors on arm have also |
| | | decreased considerably in size. Patient also |
| | | reports that toenail fungus is clearing up (has had |
| | | it for 10 years) and prostate/urinary leaking has |
| | | decreased. |
| 52 | Melanoma-stage 4 | Started the paw paw in November 2002. Felt lethargic |
| | | and had a poor appetite when he first started the |
| | | capsules. Had gradual improvement. Passed |
| | | away on May 12, 2002, but lived 14 months |
| | | longer than the doctors had predicted. |
| 53 | Melanoma-stage 4 | Passed away, brain metastases. |
| 54 | Multiple Myeloma | December 2002 start; continues to take the paw paw. |
| 55 | Neck and head | Has not been faithful in taking product. |
| | cancer | |
| 56 | Neck cancer | Shrinkage of tumor in 1 month, but passed away |
| | | from hemorrhage in neck. |
| 57 | Ovarian Cancer | January 2003 start. |
| 58 | Ovarian Cancer | Stopped taking product because of illness. May |
| | | start again as patient begins feeling better. |
| 59 | Ovarian Cancer | Had surgery for tumor near back causing pain |
| | | and radiation. Will continue paw paw. |
| 60 | Pancreatic cancer | October 2002 start; progressed. |
| 61 | Pancreatic cancer | Chemotherapy treatments caused nausea. Is not |
| | | using the paw paw because it compounded the |
| | | problem. |
| 62 | Prostate cancer | December 2002 start. PSA (prostate specific antigen) |
| | | went up to 10.5 (Mar. 7, 2003) from 5.5 (December 2002). |
| 63 | Prostate cancer | Could not tolerate. |
| 64 | Prostate cancer | Started the paw paw in October 2002. PSA |
| | | dropped to 2.08 on Dec. 23, 2002 down from a PSA of |
| | | 3.85 taken two months previously and today |
| | | continues to remain at normal PSA levels. |
| 65 | Prostate cancer | Started in January. PSA rising slightly. |
| 66 | Prostate cancer | Started the paw paw in January 2003. The most |
| | | recent prostate exam showed that prostate was |
| | | not enlarged and in normal condition. This is the |
| | | first time in 10 years it has not been enlarged. |
| 67 | Prostate cancer | Has been taking the paw paw since January of |
| | | 2002. PSA continues to be stable over the |
| | | course of the year: 8.7 on Feb. 14, 2002, 7.6 on |
| | | Apr. 15, 2002, 8.2 on Jun. 14, 2002, 7.9 on Aug. 15, 2002, 8.5 on |
| | | December 2002, without any other treatments. |
| 68 | Prostate cancer | Started the paw paw in October 2002. PSA is |
| | | very low at 0.58 (on Dec. 27, 2002, down from 6.9 |
| | | Sep. 27, 2002) and has stayed low with the paw paw. |
| | | Has also been taking Lupron shots monthly. |
| 69 | Prostate cancer | Had surgery and baseline PSA is 2.5. Will start |
| | | in January. |
| 70 | Prostate cancer | Has been taking product since January 2003. The |
| | | PSA has fluctuated but went up to 175, |
| | | previously at 155. |
| 71 | Prostate cancer | Started the product in January 2003. The cancer has |
| | | metastasized to brain. An MRI done on |
| | | Mar. 11, 2003 showed some regression in 8 |
| | | brain lesions. |
| 72 | Prostate cancer | Started the paw paw in August 2002. Has been |
| | | doing well and the PSA levels are staying |
| | | constant (7.7, 6.3, and 7.9 in November 2002, recently |
| | | 6.2). |
| 73 | Prostate cancer | Has been using the paw paw with chemotherapy. |
| | | PSA has slightly climbed to 101. Most recent |
| | | results have not been obtained. |
| 74 | Prostate cancer | Started the paw paw capsules in January 2003. PSA |
| | | has decreased slightly (PSA on Mar. 11, 2003 was 6.1, |
| | | down from 7.7 in December 2002). |
| 75 | Prostate cancer | Feb 1 started taking product. Will continue and |
| | | see doctor in a couple of months. |
| 76 | Prostate cancer | Started paw paw in November 2002. PSA taken on |
| | | Jan. 2, 2002 lowered to 1.7, which is down from 4.5 a |
| | | couple months ago. Bone scan was also normal. |
| 77 | Prostate cancer | Starting in March 2003, wanted to wait to get |
| | | base PSA reading. |
| 78 | Prostate cancer | Started taking the paw paw in January 2003 and has |
| | | been doing very well. PSA levels are currently |
| | | very low. Has been using hormonal therapy |
| | | (Lupron) as well as paw paw. |
| 79 | Prostate cancer | Stopped taking product due to unrelated health |
| | | problems and nausea. Did take the product for |
| | | 4-5 months and reported feeling good and having |
| | | more energy. |
| 80 | Prostate cancer | Started the paw paw in October 2002. PSA levels |
| | | have been stable. |
| 81 | Prostate cancer | Started the paw paw in October 2002. PSA levels are |
| | | remaining constant (PSA results 7.82, 7.62, and |
| | | 7.6). |
| 82 | Prostate cancer | December 2002 start; has only been taking one capsule a |
| | | day. |
| 83 | Prostate cancer | MD reports that the PSA levels are decreasing. |
| 84 | Prostate cancer- | Has been taking the paw paw since August 2002. |
| | stage 4 | Has multiple metastases in neck, groin, and |
| | | retroperitoneal areas. The Oct. 3, 2002 CT scan |
| | | showed “distinct regression in tumor masses |
| | | which was a 25% reduction” compared to Jul. 8, 2002 |
| | | CT scan. Currently the PSA levels are stable. |
| 85 | Rectal cancer | Waited until March 2003 to start taking paw paw. |
| 86 | Rectal cancer | MD reports that patient is clinically stable. |
| 87 | Squamous cell | January 2003 start; will be sending in test results |
| | carcinoma | shortly. |
| 88 | Squamous cell | January 2003 start. |
| | carcinoma | |
| 89 | Stomach cancer | 4 months of capsules, partial control of disease. |
| 90 | Throat | January 2003 start. |
| 91 | Uterine cancer | January 2003 start. Recently had surgery and |
| | | radiation. |
| 92 | Uterine cancer | Started the paw paw in August 2002. Patient |
| | | reports that feels more energy taking the paw |
| | | paw, but has not been completely well. Passed |
| | | away on Jan. 28, 2003 but the paw paw possibly |
| | | gave several extra months to live. |
| 93 | Uterine cancer | Has been taking for 2 months. Taking Doxil |
| | | chemotherapy since ca 125 levels have slightly |
| | | increased. |
| 94 | Uterine/cervical | Started the paw paw in November 2002. Was feeling |
| | | lethargic and had a poor appetite at first but it |
| | | improved by December. Passed away on |
| | | Mar. 17, 2003. |
| 95 | | MD reports deteriorating condition. |
| 96 | | Stable condition according to MD. |

According to the study, levels of PSA were held constant and even decreased. Likewise, levels of breast tumor antigens were significantly reduced. Tumor sizes, e.g., in breast cancer, lymphomas, and melanomas, decreased and some have even disappeared. Adverse effects were practically nonexistent with this regime of QID supplement servings over 10 or more months. The capsules are safe, effective and helpful at all stages of several types of clinical malignant cancer and are a benefit with or without chemotherapy, surgery and/or radiation.

The present inventions may be embodied in other specific forms without departing from their spirit. The described embodiments are to be considered in all respects only as illustrative and not restrictive. The scope of the inventions is, therefore, indicated by the appended claims rather than by the foregoing description.

1-14. (canceled)

15: A method for treating cancer, comprising administering to a patient a therapeutically effective amount of a crude extract containing annonaceous acetogenin compounds from a plant of Asimina triloba.

16: The method of claim 15, wherein the method of treating cancer comprises administering a therapeutically effective amount of the crude extract to a patient diagnosed with bone cancer.

17: The method of claim 16, wherein the therapeutically effective amount of the crude extract is sufficient to decrease a level of alkaline phosphatase in the blood.

18: The method of claim 15, wherein the method of treating cancer comprises administering a therapeutically effective amount of the crude extract to a patient diagnosed with breast cancer.

19: The method of claim 18, wherein the therapeutically effective amount of the crude extract is sufficient to decrease a level of tumor marker CA2729.

20: The method of claim 15, wherein the method of treating cancer comprises administering a therapeutically effective amount of the crude extract to a patient diagnosed with lung cancer.

21: The method of claim 20, wherein the therapeutically effective amount of the crude extract is sufficient to decrease a level of carcinoembryonic antigen (CEA) in the blood.

22: The method of claim 15, wherein the method of treating cancer comprises administering a therapeutically effective amount of the crude extract to a patient diagnosed with prostate cancer.

23: The method of claim 22, wherein the therapeutically effective amount of the crude extract is sufficient to decrease a level of prostate specific antigen (PSA) in the blood.

24: The method of claim 15, further comprising co-administering to the patient a therapeutically effective amount of a chemotherapeutic agent.

25: A method for inducing tumor reduction, comprising administering to a patient a therapeutically effective amount of a crude extract containing annonaceous acetogenin compounds from a plant of Asimina triloba.

26: The method of claim 25, wherein administering to a patient a therapeutically effective amount comprises administering the crude extract in capsule form four times daily.

27: The method of claim 26, wherein administering to a patient a therapeutically effective amount comprises administering about 12.5 milligrams of the crude extract in capsule form four times daily.

28: The method of claim 26, wherein administering to a patient a therapeutically effective amount comprises administering the crude extract to the patient for a period of at least 180 days.

29: The method of claim 25, further comprising co-administering to the patient a therapeutically effective amount of a chemotherapeutic agent.

30: A method for improving the wellness of a human subject, comprising administering to the human subject a crude extract containing annonaceous acetogenin compounds from a plant of Asimina triloba.

31: The method of claim 30, wherein administering the crude extract comprises administering the crude extract to the human subject in capsule form four times daily.

32: The method of claim 31, wherein administering the crude extract comprises administering about 12.5 milligrams of the crude extract in capsule form to the human subject four times daily.

33: The method of claim 31, wherein administering the crude extract comprises administering the crude extract to the human subject for a period of at least 180 days.
